(19) United States
(12) Plant Patent Application Publication
(10) Pub. No.: US 2012/0291163 P1
(43) Pub. Date: Nov. 15, 2012

(54) PETUNIA PLANT NAMED 'DLITT258'

(50) ;

(76) Inventor: Gavriel Danziger, Moshav Mishmar Hashiva (IL)

(21) Appl. No.: 13/068,514

(22) Filed: May 13, 2011

(51) Int. Cl. A01H 5/00

(52) U.S. Cl. PLT/356.18

(57) ABSTRACT

A new and distinct Petunia cultivar named ‘DLITT258’ is disclosed, characterized by a compact plant habit. The new variety has a distinctive, small, purple flowers with darker veins. The new variety is a Petunia, normally produced as an outdoor garden or container plant.

### LATIN NAME OF THE GENUS AND SPECIES

Petunia sensu Wijsman

### VARIETY DENOMINATION

‘DLITT258’

### BACKGROUND OF THE INVENTION

The new Petunia cultivar is a product of a planned breeding program conducted by the inventor, Gavriel Danziger, in Moshav Mishmar Hashiva, Israel. The objective of the breeding program was to produce new Petunia varieties for ornamental commercial applications. The cross resulting in this new variety was made during November 2008.

The seed parent is the unpatented, proprietary seedling variety referred to as Petunia sensu Wijsman ‘Pe-3031’ The pollen parent is the unpatented proprietary seedling variety referred to as Petunia sensu Wijsman ‘Pe-169’. The new variety was discovered in March 2009 by the inventor in a group of seedlings resulting from the 2008 crossing, in a research greenhouse in Moshav Mishmar Hashiva, Israel.

Asexual reproduction of the new cultivar ‘DLITT258’ by vegetative cuttings was first performed at a research greenhouse in Moshav Mishmar Hashiva, Israel in March 2009 and has shown that the unique features of this cultivar are stable and reproduced true to type in successive generations.

### SUMMARY OF THE INVENTION

The cultivar ‘DLITT258’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DLITT258’ These characteristics in combination distinguish ‘DLITT258’ as a new and distinct Petunia cultivar:

1. Compact plant habit

2. Small flowers

3. Distinctive purple flowers with dark veins.

Plants of the new cultivar ‘DLITT258’ are similar to plants of the seed parent, Petunia sensu Wijsman ‘Pe-3031’ in most horticultural characteristics, however, plants of the new cultivar ‘DLITT258’ produce more lateral branches. Additionally, the seed parent produces white flowers whereas ‘DLITT258’ produces purple flowers with darker veins.

Plants of the new cultivar ‘DLITT258’ are similar to plants of the pollen parent, Petunia sensu Wijsman ‘Pe-169’ in most horticultural characteristics, however, plants of the new cultivar ‘DLITT258’ produce, smaller flowers. Additionally, the new variety produces purple flowers with darker veins whereas the pollen parent produces blue flowers.

### COMMERCIAL COMPARISON

Plants of the new cultivar ‘DLITT258’ are comparable to the variety Petunia ‘DANCASDEEP’ U.S. Plant Pat. No. 17,020. The two Petunia varieties are similar in most horticultural characteristics however, the new variety ‘DLITT258’ differs in having purple flowers, compared to red flowers on this comparator variety. Additionally ‘DLITT258’ produces much smaller flowers and more lateral branches than ‘DANCASDEEP’. Plants of ‘DLITT258’ have a compact plant habit, whereas plants of ‘DANCASDEEP’ have a trailing plant habit.

Plants of the new cultivar ‘DLITT258’ are comparable to the unpatented variety Petunia ‘Cascadias White’. The two Petunia varieties are similar in most horticultural characteristics however, the new variety ‘DLITT258’ differs in having purple flowers, compared to white flowers on this comparator variety. Additionally ‘DLITT258’ produces smaller flowers and slightly more lateral branches than ‘DANCASDEEP’.

### BRIEF DESCRIPTION OF THE PHOTOGRAPHS

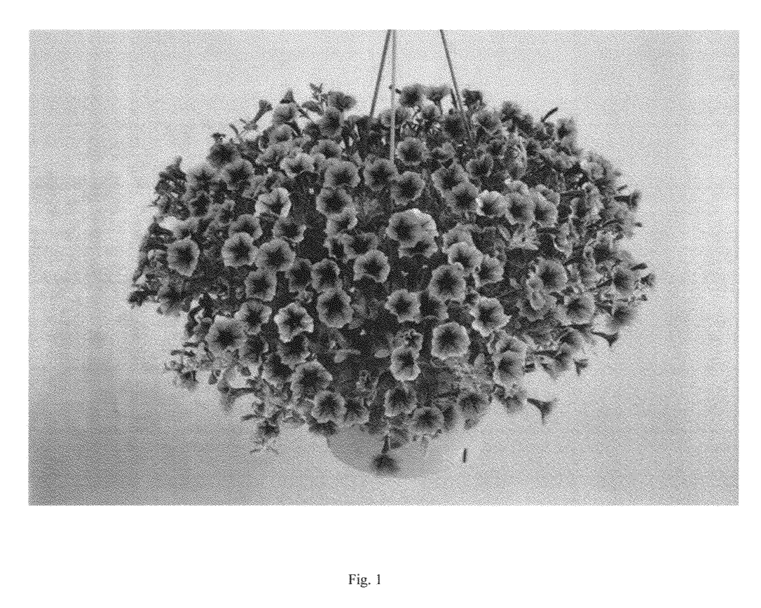

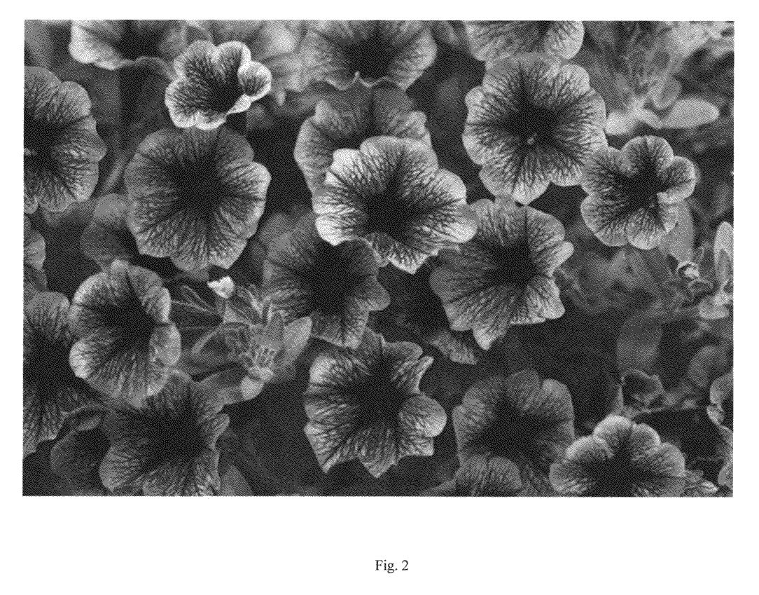

The accompanying photograph in FIG. 1 illustrates in full color a typical plant of ‘DLITT258’ grown in a greenhouse, in a 13 cm pot. Age of the plant photographed is approximately 7 weeks from a rooted cutting. FIG. 2 illustrates in full color a close up of a typical bloom of ‘DLITT258’. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001 except where general terms of ordinary dictionary significance are used. The following observations and measurements describe ‘DLITT258’ plants grown outdoors during Autumn in Moshav Mishmar Hashiva, Israel. The growing temperature ranged from 20° C. to 35° C. during the day and from 17° C. to 23° C. during the night. General light conditions are bright, normal sunlight. Measurements and numerical values represent averages of typical plant types.
Botanical classification: Petunia sensu Wijsman ‘DLITT258’
Propagation:
   Time to initiate roots.—10 to 14 days at approximately 18-35° C.
   Root description.—fibrous.
Plant:
   Age of plant described.—Approximately 30 days.
   Growth habit.—Compact.
   Pot size of plant described.—12 cm.
   Height.—10 cm.
   Plant spread.—20 cm.
   Growth rate.—Rapid.
   Branching characteristics.—Very highly branched.
   Length of primary lateral branches.—10 cm.
   Diameter of lateral branches.—0.1 cm.
   Quantity of primary lateral branches.—25-30.
   Characteristics of primary lateral branches.—Diameter: 0.3 cm. Color: Near RHS Green 139-A Texture: Pubescent Strength: Flexible.
   Internodes length.—2-4 cm.
Foliage:
   Leaf.—Leaf: Arrangement: Opposite Quantity: Approximately 10 per branch. Average Length: 3 cm. Average Width: 1.5 cm. Shape of blade: Oval Apex: Rounded Base: Acute Margin: Entire Pubescence: Pubescent Angle of attachment: 90° Aspect: Flat Color: Young foliage upper side: RHS 139-A Young foliage under side: RHS 139-A Mature foliage upper side: RHS 139-A Mature foliage under side: RHS 139-A Venation: Type: Pinnate Venation color upper side: RHS 139-A Venation color under side: RHS 139-A Petiole: Not present.
Flower:
   Natural flowering season.—All year in Mediterranean climates.
   Days to flowering from rooted cutting.—10-20 days.
   Inflorescence and flower type and habit.—Terminal, Single, Salver form, Erect.
   Rate of flower opening.—2 to 3 days from bud to fully opened flower.
   Flower longevity on plant.—3-6 days.
   Approximate quantity of flowers per plant.—Approximately 30.
   Persistent or self-cleaning.—self-cleaning.
   Bud.—Shape: tubular Length: 2 cm Diameter: 0.3 cm Color RHS: Greyed-Purple N187B.
   Flower size.—Diameter: 2.5 cm. Flower tube length: 2.5 cm. Flower tube diameter at distal end: 1 cm. Flower tube diameter at proximal end: 0.2 cm.
   Petals.—Length from throat: 1 cm. Width: 1.4 cm. Quantity: 5 Texture: Smooth Apex: Blunt Margin: Entire.
   Color.—When opening: Upper surface: Near RHS Violet 86C with veins 86A Lower surface: Near RHS Violet 86C with veins 86B Fully opened: Upper surface: Near RHS Violet 86C with veins 86A Lower surface: Near RHS Violet 86C with veins 86B Flower throat (inside): Near RHS Purple 79A Flower throat, veins: Near RHS Purple 79A Flower tube (outside): Near RHS Purple 79B Flower tube, veins: Near RHS Purple 79B Fading Petals fading to: Near RHS Purple 79C.
   Sepals.—Quantity per flower: 5 Shape: Elliptic Length: 1 cm Width: 0.1 cm Apex: Truncate Base: Cuneate Margin: Entire Texture: Lustrous Color: Upper Surface: Near RHS Green 139A Lower Surface: Near RHS Green 139A.
   Peduncle.—Length: 2.5 cm. Diameter: 0.1 cm. Color: RHS Green 139-A Orientation: 45 degree, straight.
   Fragrance.—No.
Reproductive organs:
   Stamens.—Number: 5 Filament length: 1 cm
   Anthers.—Length: 0.1 cm Shape: Round Color: RHS Yellow 11-A Pollen: Color: RHS Violet-Blue 94B Quantity: Abundant.
   Pistil.—Number: 1 Length: 1 cm. Style: Length: 1 cm. Color: RHS Green 139-C Stigma: Shape: Round Color: RHS Green 139-C Ovary Color: RHS Green 139-A.
Other characteristics:
   Seeds and fruits.—Numerous minute dark seeds.
   Disease/pest resistance.—Neither resistance nor susceptibility to pathogens and pests common to Petunia have been observed.
   Temperature tolerance.—The new variety tolerates temperatures between 5 to 40° C.

What is claimed is:

1. A new and distinct cultivar of Petunia plant named ‘DLITT258’ as herein illustrated and described.
